(19) United States
(12) Patent Application Publication
(10) Pub. No.: US 2004/0010185 A1
(43) Pub. Date: Jan. 15, 2004

(54) Method for measuring a physiologic parameter using a preferred site

(75) Inventors: Victor E. Kimball, Burnsville, MN (US); Steven C. Furlong, Maple Grove, MN (US); Irvin Pierskalla, Prior Lake, MN (US)

Correspondence Address: ALTERA LAW GROUP, LLC, 6500 CITY WEST PARKWAY, SUITE 100, MINNEAPOLIS, MN, 55344-7704, US

(73) Assignee: Optical Sensors, Inc., Minneapolis, MN (US)

(21) Appl. No.: 10/195,005

(22) Filed: Jul. 11, 2002

(51) Int. Cl. A61B 5/00

(52) U.S. Cl. 600/310

(57) ABSTRACT

Generally, the present invention relates to a method for non-invasive optical measurements at at physiologic sites that may reduce or minimize the effects of skin chemistries that optically interfere with the desired optical measurement. An embodiment of the invention is directed to a method of making an optically-based, non-invasive optical measurement of a first physiologic parameter of a patient. The method comprises probing the tissue of a first epithelial site with a first probe light propagating from the optical sensor and detecting a first signal light received from the first assay site with the optical sensor. The method also comprises measuring a value of a second parameter of the patient and determining the level of the first physiologic parameter within the tissue of the first assay site based on the detected first signal light and on the measured second parameter of the patient.

### FIELD OF THE INVENTION

The present invention is directed generally to medical devices and more particularly to non-invasive optical sensors for physiologic parameters and a preferred patient site for such measurements.

### BACKGROUND

Optical spectroscopy techniques have been developed for a wide variety of uses within the medical community. For example, pulse oximetry and capnography instruments are in widespread use at hospitals, both in the surgery suites and the post-op ICU's. These technologies have historically been based on absorption-based spectroscopy techniques and have typically been used as trend monitors in critical care environments where it is necessary to quickly determine if a patient's vital parameters are undergoing large physiologic changes. Given this operating environment, it has been acceptable for these devices to have somewhat relaxed precision and accuracy requirements, given the clinical need for real-time point-of-care data for patients in critical care situations.

Both pulse oximeters and capnography instruments can be labeled as non-invasive in that neither require penetrating the outer skin or tissue to make a measurement, nor do they require a blood or serum sample from the patient to custom calibrate the instrument to each individual patient. These instruments typically have pre-selected global calibration coefficients that have been determined from clinical trial results over a large patient population, and the results represent statistical averages over such variables as patient age, sex, race, and the like.

There is, however, a growing desire within the medical community for non-invasive instruments for use in such areas as the emergency room, critical care ICU's, and trauma centers where fast and accurate data are needed for patients in potentially life threatening situations. One such measurement needed in these environments is the blood and/or tissue pH level, which is a measure of the free hydrogen ion concentration. This is an important measure of intracellular metabolism. Biological processes within the human body require a narrow range of pH for normal function, and significant changes of pH from this range may be life threatening.

In addition to pH, it is also typical for other physiologic parameters such as the blood gases (O₂ & CO₂), blood electrolytes, cardiac-event enzyme markers, and other blood chemistry parameters such as glucose, to be measured and monitored during critical care treatment. Technologies for making these measurements have been in place for nearly fifty years in hospital laboratories. These measurements are made from blood samples drawn from the patient which are then sent to a laboratory for analysis. These laboratory measurements are typically made with electrochemical sensors.

Recent developments in non-invasive optical technology hold the potential that some of these measurements may be made at the point of care with sufficient precision and accuracy to carry out critical care monitoring and treatment. Also, there has been an increased interest in utilizing both the absorbance and fluorescence properties of naturally occurring biological molecules as physiologic markers for non-invasive optical measurements. Both of these techniques are complicated by the patient-to-patient variability in skin texture and chemical composition, both of which affect the optical properties of the skin and make universal calibration of such devices difficult.

### SUMMARY OF THE INVENTION

Given the situation described above there is a need for a technique to custom calibrate non-invasive optical physiologic sensors to each individual patient. In particular, it may also be beneficial to have a physiologic site for non-invasive optical measurements that reduces or minimizes the effect of skin chemistries that optically interfere with the desired optical measurement. Such a technique may be applicable to a wide variety of commonly monitored physiologic parameters during critical care patient management.

One particular embodiment of the invention is directed to a method of making an optically-based, non-invasive optical measurement of a first physiologic parameter of a patient. The method comprises probing the tissue of a first epithelial site with a first probe light propagating from the optical sensor and detecting a first signal light received from the first assay site with the optical sensor. The method also comprises measuring a value of a second parameter of the patient and determining the level of the first physiologic parameter within the tissue of the first assay site based on the detected first signal light and on the measured second parameter of the patient.

The above summary of the present invention is not intended to describe each illustrated embodiment or every implementation of the present invention. The figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 illustrates a facial view of a patient depicting the preferred physiologic sites for spectroscopic tissue assessment.

FIG. 2 illustrates a schematic representation of a non-invasive physiologic monitoring device.

FIG. 3 illustrates a cross-sectional view of a patient depicting the trachea as a physiologic site for optical spectroscopy.

FIG. 4 illustrates a cross-sectional view of a patient depicting the esophagus as a physiologic site for optical spectroscopy.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is applicable to medical devices and is believed to be particularly useful for non-invasive optical physiologic sensors.

Generally, the present invention relates to a method of measurement and to preferred physiologic sites to perform non-invasive fluorescent spectroscopy on human tissue. In addition to fluorescent spectroscopy, other optical measurement techniques such as absorbance spectroscopy, both in transmission and reflectance, or photon migration spectroscopy may be utilized separately or in conjunction with fluorescent measurement techniques. The sites may be accessed in a non-invasive manner without surgical procedures and it may be possible to both non-invasively calibrate and perform assay measurements at the same physiologic sites. Approaches to non-invasively calibrate optical physiologic sensors are discussed in U.S. patent application Ser. No. ______ titled, “Calibration Technique For Non-Invasive Optical Medical Devices”, by inventors Victor Kimball, Steven Furlong, and Irvin Pierskalla, Altera Law Group Docket # 1535.1US01, filed on even date herewith, which is incorporated herein by reference. Also, approaches to non-invasively optically measure CO₂ concentrations are discussed in U.S. Pat. No. 6,055,447 titled, “Patient CO₂ Measurement”, by inventors Max Weil, Wanchun Tang, and Jose Bisera, and approaches to non-invasively optically measure pH is discussed in U.S. patent application Ser. No. ______ titled, “Non-invasive Measurement of pH”, by inventors Victor Kimball, Steven Furlong, and Irvin Pierskalla, Altera Law Group Docket # 1535.2US01, filed on even date herewith, both of which are also incorporated herein by reference.

In some cases, glucose for example, it may be beneficial to measure additional physiologic parameters or characteristics of physiologic parameters at the same physiologic site in order to increase the accuracy of the measurement. Similarly, it may be beneficial in some cases to measure the additional physiologic parameters simultaneously with the main physiologic measurement. An example of this technique is described in U.S. Pat. No. 5,672,515 titled, “Simultaneous Dual Excitation/ Single Emission Fluorescent Sensing Method For pH and pCO₂”, by inventor Steven Furlong, which is incorporated herein by reference. Other examples of physiologic parameters whose measurement may benefit from this technique are hemoglobin and bilirubin. Specifically, when measuring the hemoglobin fractions, say oxy-hemoglobin and carboxy-hemoglobin, it may prove beneficial to make the measurements in tandem to increase the accuracy in ultimately calculating the remaining hemoglobin fractions.

Many physiologic parameter measurements can benefit from the measurement or determination of a second physiologic parameter. For example, when measuring the partial pressure of dissolved oxygen (pO₂) in blood, it is useful to also measure the blood temperature to compensate for the hemoglobin affinity to oxygen, which is temperature dependent, and modulates the available supply of free oxygen dissolved in blood. One approach of measuring blood pO₂ is made using immobilized fluorescent O₂ indicators on the distal end of optical fibers immersed in the blood and calibrating the response of the fluorescent indicators to precise pO² levels. Temperature measurements of the environment near the distal end of the pO₂ sensing optical fiber can be measured by standard thermocouples or thermistors.

Similarly, the accuracy of in-vivo glucose measurements are enhanced by a blood or interstitial fluid (ISF) temperature measurement to compensate for the large temperature dependence of the water absorption bands which might otherwise obscure the glucose signal. For example, glucose measurements can be made optically in either the fluorescent or absorption mode. In the absorption mode, it is typical that near infrared optical radiation is delivered to the patient to measure the glucose absorption, which is concentration dependent. The same infrared energy heats the water molecules in blood, altering the H₂O absorption which might corrupt or interfere with the glucose measurement. Typically, the blood/ISF temperature is simultaneously measured and an algorithmic compensation is made to the glucose calculation to compensate for the H₂O temperature dependence.

Bilirubin and hemoglobin measurements can also benefit from the measurement or determination of a second physiologic parameter. For example, both bilirubin and hemoglobin can be measured optically by absorption techniques. Tissue constituents collagen, elastin, and melanin all are known to absorb optical radiation in wavelength bands which might interfere with the primary measurement of bilirubin/hemoglobin. In these cases, it is typical to pick specific wavelength regions intrinsic to the determination of the concentration of the secondary physiologic parameter to algorithmically compensate for their effect on the primary measurement. Another secondary physiologic parameter which may prove beneficial is the volume being assayed in a non-invasive measurement. One example of this would be the non-invasive measurement of hematocrit, wherein the volume of the measurement may be determined from the geometrical relationship between the optical source and detector(s).

Non-invasive optical measurements of blood pH and CO₂ may also benefit from secondary measurements which increase the accuracy of the primary measurement. The non-invasive measurement of pH can be made by measuring the induced fluorescence of naturally occurring biological markers which are sensitive to the local pH environment. The fluorescence quantum efficiency of these biological markers is also temperature dependent and isolating the temperature response from the pH response can lead to a more accurate pH determination. In addition to compensating for temperature-induced effects when non-invasively measuring pH, it may prove beneficial to also optically measure a second fluorescent biological marker which is pH insensitive. The above approach is advantageous in situations where the two fluorescent species can be excited by the same optical source utilizing the same excitation optical pathway, in this configuration common-mode error mechanisms such as light source fluctuations and mechanical or vibration induced misalignments can be suppressed by ratio'ing techniques. An example of this technique is described in U.S. patent application Ser. No. ______ titled, “Non-invasive Measurement of pH”, by inventors Victor Kimball, Steven Furlong, and Irvin Pierskalla, Altera Law Group Docket # 1535.2US01, filed on even date herewith, which is incorporated herein by reference.

Non-invasive optical CO₂ measurements are routinely made in hospital ICU environments as near infrared absorption spectroscopy of end-tidal expired gas. Here too, the primary physiologic measurement can be augmented by secondary absorption spectroscopy of interfering species such as nitrous oxide (N₂O), carbon monoxide (CO), and expired water vapor (H₂O) all of which have residual absorption near the CO₂ absorption peak at 4.26 microns.

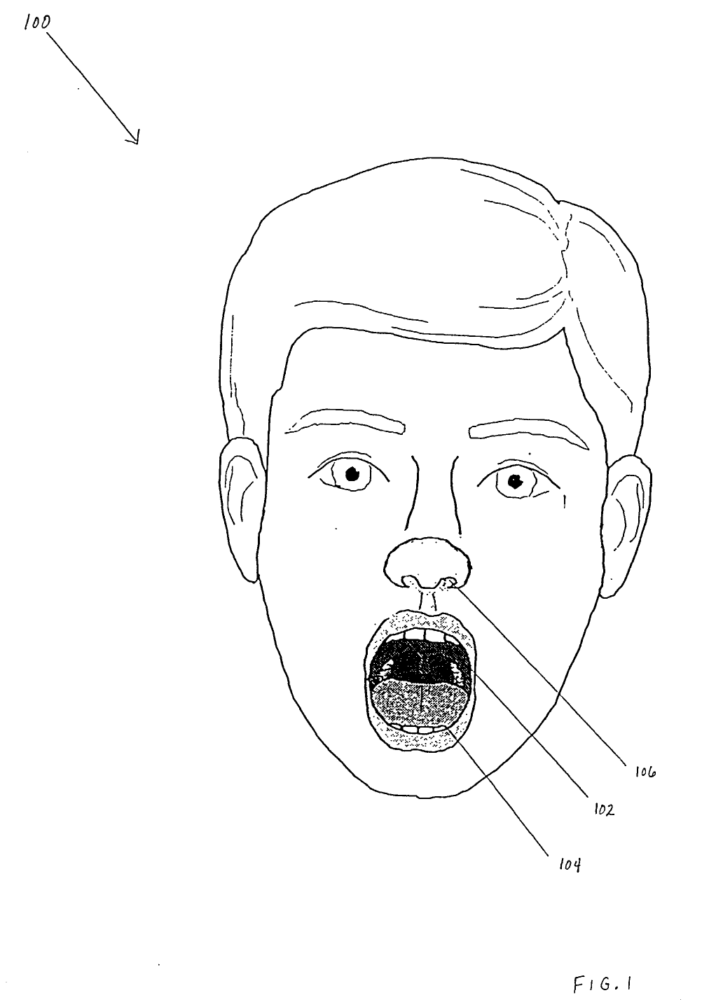

FIG. 1 illustrates a facial view 100 of a patient depicting some of the preferred physiologic sites 102, 104, and 106 for non-invasive physiologic measurements. All three physiologic sites have in common the lack of the skin pigmentation component melanin which may have optical properties which would otherwise interfere with the optical measurements. Also, the three sites are composed of epithelial tissue devoid of major arteries or veins, mostly being nourished via the tissue capillary bed, thereby possibly reducing the effects of interference due to localized hemoglobin absorption. The epithelial tissue is composed of cells which line the body cavities and the principal tubes and passageways leading to the exterior. In addition to the possible decreased hemoglobin concentration, the three sites may have a spatially uniform distribution of physiologic parameters in the tissue bed, thereby reducing the sensitivity of the tissue measurements to the placement of the physiologic sensors. The three physiologic sites may also have a shorter optical path length and concomitant lower optical absorption/scatttering to the physiologic parameters than, for example the fingertip region commonly used for pulse oximetry, due to the capillary bed being closer to the epithelial surface in the preferred physiologic sites. Given the proximity of the capillary bed to the epithelial surface at the preferred physiologic sites, it may be beneficial to differentiate the pulsatile optical response, indicative of the blood-borne concentration of the physiologic parameters, from the low frequency (non-pulsatile) response indicative of the tissue bed concentration.

Site 102, the inner lining of the cheek (sometimes referred to as the buccal region) is readily accessible for physiologic measurements and sensors may be attached to the inner cheek lining via appropriate retaining devices for monitoring in an ICU or emergency room type environment. Appropriate retaining devices or techniques may include clips, handles, immobilizing the optical sensor between clenched teeth, utilizing an inflatable balloon device to stabilize the sensor against the cheek lining or other suitable techniques.

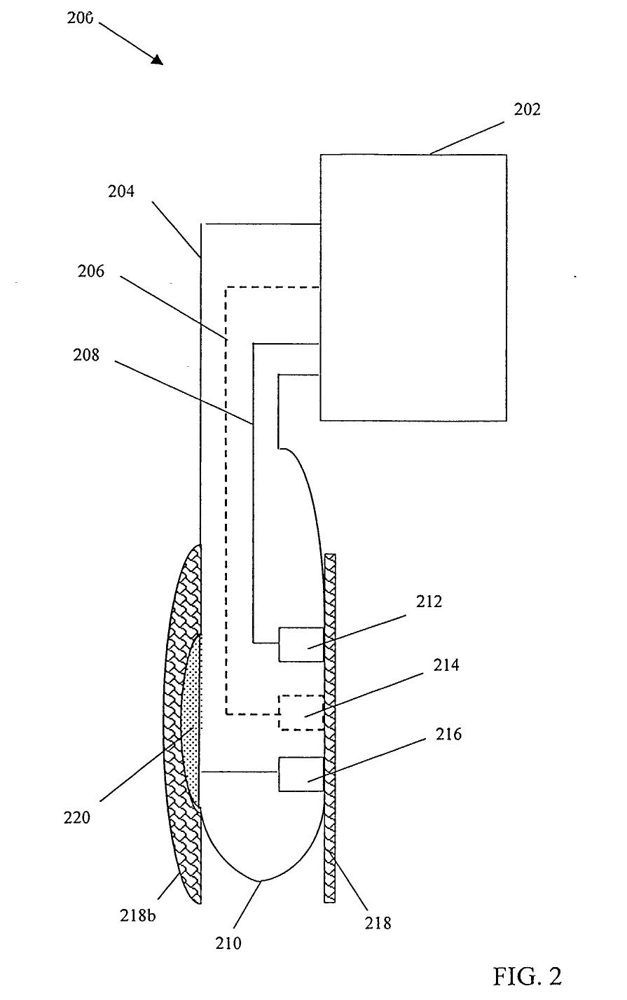

Site 104, under the tongue (sublingual) and site 106 in the nostrils (sometimes referred to as the nares region) are also readily accessible for non-invasive sensors. Both sites 102 and 104 may be utilized with the non-invasive physiologic monitoring device 200 depicted in FIG. 2, wherein the calibration and follow-on assay measurements are performed at substantially the same location. The physiologic monitoring device 200 is described in further detail in U.S. patent application titled, “Calibration Technique For Non-Invasive Optical Medical Devices”, Altera Law Group Docket # 1535.1US01. Sites 102 and 104 are suited for this common calibration/assay site in that both locations have a back surface for the inflatable bladder 220 as described below.

An embodiment of a non-invasive physiologic monitoring device 200 is depicted in FIG. 2. This embodiment may be particularly useful for conducting assays in a lumen, such as the esophagus, trachae, or rectal regions. A processor/controller module 202 may contain the electro-optic sub-systems and a central processing unit to control the timing, delivery, routing and post processing of signals for the monitoring device 200. An optical interface 204 connects the controller module 202 to a first non-invasive optical physiologic sensor 212, which may be housed in a patient interface module 210. The optical interface 204 may be a fiber optic waveguide or a fiber optic bundle, or discrete bulk optical components such as a condensing lens or a series of condensing lenses. The patient interface module 210 may provide protection from such unwanted outside influences as stray light, fluid spills, and the like. The first non-invasive optical physiologic sensor 212 may be in direct physical contact with the patient's epithelial tissue surface 218. The interconnect device 206 connects the controller module 202 with the stimulus transducer 214, which may also be housed in the patient interface module 210. The optical interface 208 connects the controller module 202 with a second non-invasive optical physiologic sensor 216, which may also be housed in the patient interface module 210. In this configuration, the stimulus transducer 214 and the non-invasive optical sensors 212 and 216 may be mounted sufficiently close so as to stimulate and measure the tissue response at substantially the same physical location.

An inflatable bladder 220 may be incorporated into the patient interface module 210 for those applications where the sensor is inserted into a body cavity or orifice. This embodiment is advantageous in applications where it is desirable to apply pressure from the back surface 218b of the patient's epithelial tissue surface 218b to either mechanically secure the sensor against slippage during measurement or to apply additional pressure stimulus to aid in the calibration process. Other patient interface geometries and alternative sensor configurations are described in U.S. patent application Ser. No. 10/162,028, titled “Noninvasive Detection of A Physiologic Parameter Within A Body Tissue Of A Patient” by inventors Edward J. Anderson et al, which is incorporated herein by reference.

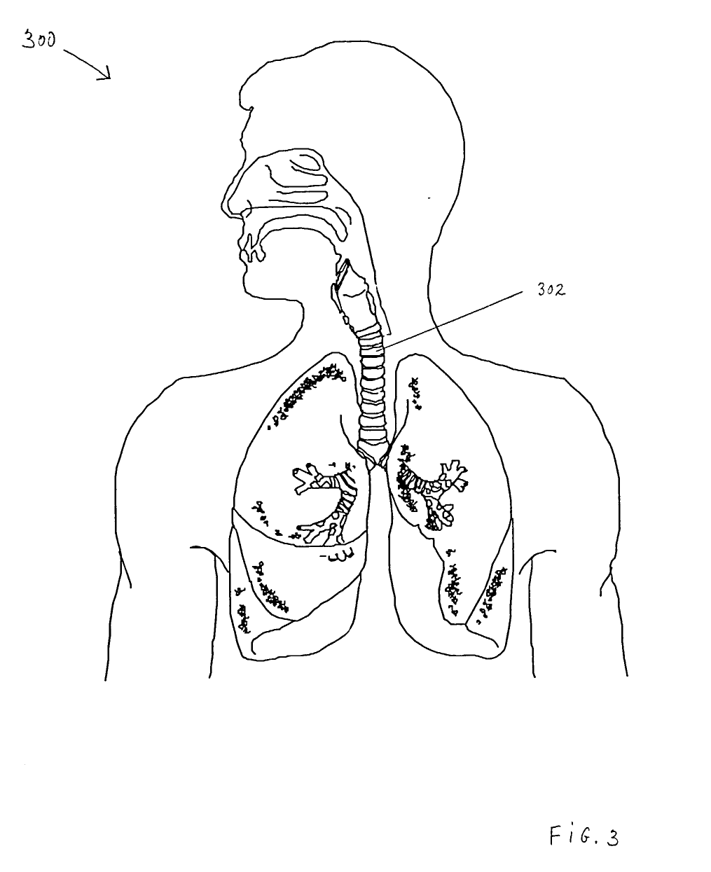

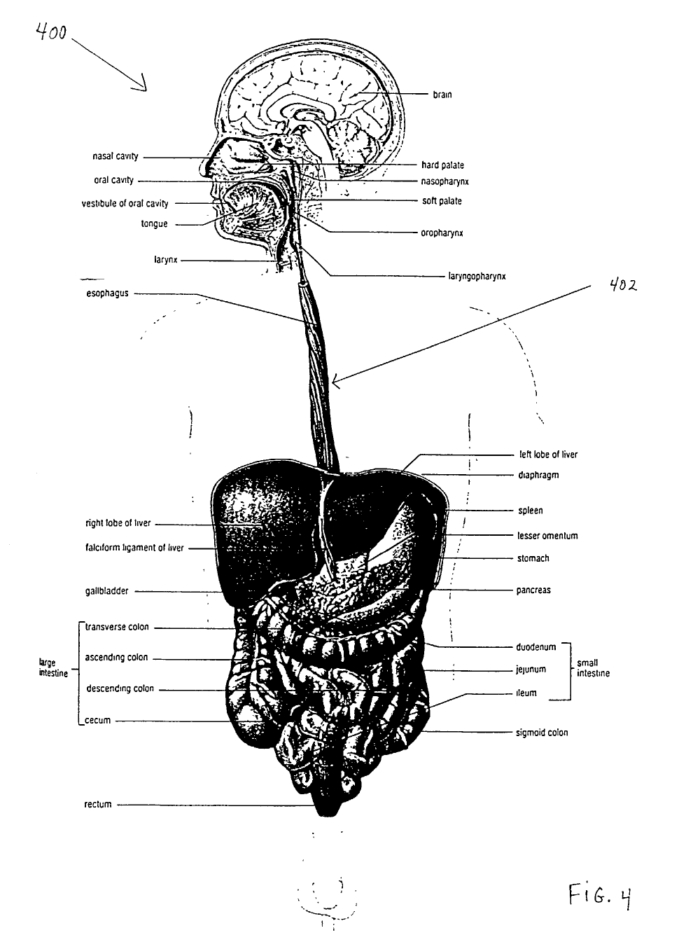

Two physiologic sites amenable to the physiologic sensor 200 described above are illustrated in FIG. 3 (for the trachae) and FIG. 4 (for the esophagus). FIG. 3 depicts a cross-sectional view 300 of a patient depicting the physiologic site 302 for optical spectroscopy of the trachae. FIG. 4 depicts depicts a cross-sectional view 400 of a patient depicting the physiologic site 402 for optical spectroscopy of the esophagus. Both sites, 302 and 402 are amenable to the inflatable bladder technique described above to either mechanically secure the sensor against slippage during measurement or to apply additional pressure stimulus to aid in the calibration process. In addition, the lower part of the large intestines (the “rectal region”) and the urinary tract leading up to and including the bladder are also amenable to the physiologic sensor 200 described earlier.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the present specification. The claims are intended to cover such modifications and devices.

I claim:

1. A method of making an optically-based, non-invasive determination of a first physiologic parameter of a patient, comprising:
probing the tissue of a first epithelial assay site with first probe light propagating from an optical sensor;
detecting first signal light received from the first assay site with the optical sensor;
measuring a value of a second parameter of the patient; and
determining the level of the first physiologic parameter within the tissue of the first assay site based on the detected first signal light and on the measured second parameter of the patient.

2. A method as recited in claim 1, further comprising measuring a value of at least a third parameter of the patient, wherein determining the level of the first physiologic parameter includes determining the level of the first physiologic parameter based on the measured value of at least the third parameter of the patient.

3. A method as recited in claim 1, wherein the first probe light optically probes an intermediate biological marker whose optical response is dependent on the first physiologic parameter.

4. A method as recited in claim 3, further comprising injecting the biological marker into the patient before probing the tissue with the first probe light.

5. A method as recited in claim 1, wherein the first probe light optically probes a biological molecule whose concentration in the tissue is the physiologic parameter.

6. A method as recited in claim 1, wherein the first epithelial assay site includes at least one of the buccal, sub-lingual, nares, esophageal, endotracheal, urinary tract, and rectal regions of the patient.

7. A method as recited in claim 1, wherein the first physiologic parameter is the tissue pH.

8. A method as recited in claim 1, wherein the first physiologic parameter is tissue pCO₂.

9. A method as recited in claim 1, wherein the first physiologic parameter is tissue pO₂.

10. A method as recited in claim 1, wherein the first physiologic parameter is one of the hemoglobin fractions.

11. A method as recited in claim 1, wherein the first physiologic parameter is total hemoglobin.

12. A method as recited in claim 1, wherein the first physiologic parameter is bilirubin.

13. A method as recited in claim 1, wherein the first physiologic parameter is hematocrit.

14. A method as recited in claim 1, wherein the first physiologic parameter is glucose concentration in the tissue.

15. A method as recited in claim 1, wherein detecting the first signal light includes detecting light at the same wavelength as the first probe light.

16. A method as recited in claim 14, wherein determining the level of the physiologic parameter includes determining the absorbance of the first probe light in the tissue based on the detected first signal light.

17. A method as recited in claim 15, wherein determining the level of the physiologic parameter includes determining the reflectance of the first probe light in the tissue based on the first detected signal light.

18. A method as recited in claim 1, wherein detecting the first signal light includes detecting a first fluorescence signal from the tissue, and determining the level of the first physiologic parameter includes determining the level of the first physiologic parameter based on the first fluorescence signal.

19. A method as recited in claim 1, wherein probing the tissue includes directing the first probe light into the tissue at a first location and detecting the first signal light includes detecting the first signal light at at least a second location of the tissue different from the first location.

20. A method as recited in claim 19, wherein detecting the first signal light further includes detecting the first signal light at a third location of the tissue different from the first and second locations.

21. A method as recited in claim 19, wherein the second and third locations are spaced apart from the first location by the same distance.

22. A method as recited in claim 19, wherein the second and third locations are spaced apart from the first location by different distances.

23. A method as recited in claim 1, determining the level of the first physiologic parameter includes determining a pulsatile value of the first physiologic parameter.

24. A method as recited in claim 1, determining the level of the first physiologic parameter includes determining a non-pulsatile value of the first physiologic parameter.

25. A method as recited in claim 1, wherein measuring the second parameter for the patient includes measuring a second physiologic parameter of the epithelial tissue.

26. A method as recited in claim 25, wherein measuring the second parameter for the patient includes probing the tissue of a second epithelial assay site with second probe light, detecting second signal light received from the second assay site and determining the level of the second physiologic parameter based at least on the detected second signal light.

27. A method as recited in claim 26, wherein the first probe light and the second probe light are at the same wavelength.

28. A method as recited in claim 26, wherein the first signal light and the second signal light are at the same wavelength.

29. A method as recited in claim 26, wherein the first signal light and the second signal light are at different wavelengths.

30. A method as recited in claim 1, wherein determining the level of the first physiologic parameter includes calibrating the optical sensor using the value of the second parameter of the patient.

31. A method as recited in claim 1, wherein determining the level of the first physiologic parameter includes calculating the level of the first physiologic parameter using the detected first signal light and the value of the second parameter of the patient.

32. A method as recited in claim 1, further comprising reporting the level of the first physiologic parameter to a user.

33. A method as recited in claim 32, further comprising reporting the value of the second parameter to the user.

34. A method as recited in claim 1, further measuring the value of the second parameter at substantially the same time as detecting the first signal light.

35. A method as recited in claim 1, further measuring the value of the second parameter at a time different from detecting the first signal light.
